(19) United States
(12) Patent Application Publication
(10) Pub. No.: US 2010/0125103 A1
(43) Pub. Date: May 20, 2010

(54) COMPOSITION FOR THE TREATMENT OF DIABETES AND METABOLIC SYNDROME CONTAINING OBOVATOL AND ITS SYNTHESIZED DERIVATIVES

(75) Inventors: Tae-Lin Huh, Daegu (KR); Hebok Song, Seoul (KR); Ji-Eun Kim, Daegu (KR); Byoung-Mog Kwon, Daejeon (KR); Dong Cho Han, Daejeon (KR); Hye-Nan Kim, Daejeon (KR)

Correspondence Address: ROTHWELL, FIGG, ERNST & MANBECK, P.C., 1425 K STREET, N.W., SUITE 800, WASHINGTON, DC, 20005, US

(73) Assignees: TG BIOTECH CO. LTD., Daegu (KR); KOREA RESEARCH INSTITUTE OF BIOSCIENCE AND BIOTECHNOLOGY, Daejeon (KR)

(21) Appl. No.: 12/484,623

(22) Filed: Jun. 15, 2009

(30) Foreign Application Priority Data
Nov. 17, 2008 (KR) 10-2008-0113991

(51) Int. Cl. A61K 31/215; C07C 43/20; A61K 31/085; C07C 69/66; A61P 3/10

(52) U.S. Cl. 514/548; 568/638; 514/721; 568/636; 560/138

(57) ABSTRACT

Disclosed is a pharmaceutical composition containing obovatol represented by Formula 1 and its derivatives. The obovatol and its derivatives effectively increase the activity of AMPK (AMP-activated protein kinase) that plays an important role in diabetes and metabolic syndrome, and thus may be variously used in treating diabetes and metabolic syndrome.

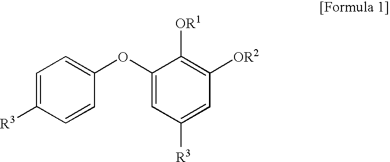

(In Formula 1, R¹, R² and R³ are the same as defined above).

### CROSS REFERENCE TO RELATED APPLICATIONS

This application claims priority from Korean patent application No. 10-2008-0113991 filed on Nov. 17, 2008, all of which is incorporated herein by reference in its entirety for all purposes.

### BACKGROUND OF THE INVENTION

1. Field of the invention

The present invention relates to a composition for preventing and treating diabetes and metabolic syndrome, which contains obovatol and its derivatives as an active ingredient. More particularly, the present invention relates to a composition for preventing and treating diabetes and metabolic syndrome, which contains obovatol and its chemically synthesized derivatives as an active ingredient, the obovatol being obtained by extracting from a magnolia ovobata fruit by using methanol, and carrying out separating and purifying processes.

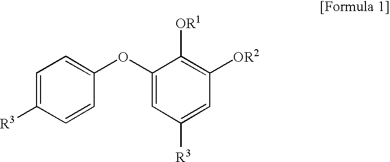

In Formula 1,

R¹ and R² independently or optionally represent hydrogen, C₁-C₇ straight chain or branched-side chain alkyl, acetyl, C₁-C₇ alkoxy or C₁-C₇ alkoxyacetyl; and

R³ represents hydrogen, C₁-C₇ straight chain or branched-side chain alkyl, C₂-C₇ alkenyl or allyl.

Preferably, R¹ and R² independently or optionally represent methyl, ethyl, acetyl, methoxy, ethoxy, methoxyacetyl or ethoxyacetyl; and

R³ represents hydrogen, propyl, propenyl or allyl.

The obovatol derivative represented by Formula 1 of the present invention specifically includes the following compounds:

1) obovatol;

2) 3-(4-propylphenoxy)-5-propylbenzene-1,2-diol (ob-RD);

3) 3-(4-propylphenoxy)-2-methoxy-5-propylphenol (ob-Rd-2M)

4) 1-(4-propylphenoxy)-2,3-diacetyloxy-5-propylbenzene (ob-Rd-Ac)

5) 1-(4-propylphenoxy)-2,3-di-2-methoxyacetyloxy-5-propylbenzene (ob-Rd-MAc)

6) 3-(4-((E)1-prop-1-enyl)phenoxy)-5-((E)-prop-1-enyl)benzene-1,2-diol (ob-RE)

7) 1-(4-((E)1-prop-1-enyl)phenoxy)-2,3-diacetyloxy-5-((E)-prop-1-enyl)benzene (ob-RE-MAc)

8) 1-(4-((E)-prop-1-enyl)phenoxy)-2,3-di-2-methoxyacetyloxy-5-(E)-prop-1-enyl)benzene (ob-RE-Ac)

2. Description of the Prior Art

[Literature 1] Hardie et al, FEBS Lett. 546, pp 113-120, (2003)

[Literature 2] David Carling et al, TRENDS in Biochemical Sciences 29, pp 18-24, (2004)

[Literature 3] Juleen R. et al, J Appl Physiol. 93, pp 773-781, (2002)

[Literature 4] Holman, R. R. et al, Metabolism, 55, S2-5,2006 ;Pickup, J. C. et al., Blackwell Scientific Publ. London, pp 462-476, (1991)

[Literature 5] Innerfield, R. J., New Engl. J. Med., 334, ₚₚ 1611-1613, (1996)

[Literature 6] Schlattner U, et al, J Biol Chem. 279, ₚₚ 43940-51, (2004)

[Literature 7] Somwar R et al, Clin Ther. 20, pp 125-40, (1998)

[Literature 8] Halseth A E et al, Biochem Biophys Res Commun. 294, pp 798-850, (2002)

### SUMMARY OF THE INVENTION

The present invention provides a composition for preventing and treating diabetes and metabolic syndrome, which contains synthesized obovatol and its single derivatives as an active ingredient.

AMPK (AMP-Activated Protein Kinase) is a kinase which plays a very important role of maintaining the energy balance in the body through the control of carbohydrate metabolism and lipid metabolism by sensing the energy state within a cell. When the content of ATP (body's energy within a cell) is reduced by intensive exercise or prolonged starvation, in other words, the ratio of ATP to AMP within a cell is significantly reduced, AMPK is activated state caused by the phosphorylation of a threonine residue at position 172 on AMPK alpha subunit. As a result, glycolysis and/or beta oxidation (β-oxidation) for generating ATP actively occur (Hardie et al, FEBS Lett. 546, ₚₚ 113-120, 2003), while inhibiting fat/cholesterol synthesis consuming ATP (David Carling et al, TRENDS in Biochemical Sciences 29, pp 18-24, 2004). Also, when AMPK is activated, blood glucose absorption is increased by several blood-sugar absorbing machinery, one of which is GLUT4 (glucose transporter 4), forwarding to cell membrane (Juleen R. et al, J Appl Physiol. 93, pp 773-781, 2002).

Particulary, it is of interesting to note that phosphorylated-activated AMPK directly phosphorylates ACC (acetyl-CoA carboxylase), an important enzyme for fatty acid synthesis, producing malonyl-CoA, and HMG-CoA reductase, the commited step enzyme in cholesterol synthesis, resulting in activity of the enzymes is reduced, thereby blocking syntheses of fat and cholesterol. Also, when available malonyl-CoA is reduced by decreased ACC activity, fatty acids are actively delivered into the mitochondria where β-oxidation (fatty acids degradation) occurs is promoted, implying accelerated lipolysis.

Metabolic syndrome was referred to as X syndrome due to its veiled cause. Recently it was found that the diminished production or malfunction of insulin, which delivers glucose to liver, muscle, or the like through glycolysis, causes various diseases, such as diabetes, hypertension, cerebral apoplexy, vascular diseases. The condition in which normal amount of insulin fails to regulate glucose refers to insulin resistance (IR), and metabolic syndrome refers to a complex disease caused by IR. Thus, the metabolic syndrome is often referred to as insulin resistance syndrome. When glucose level in the blood is not respond to insulin, and insulin concentration is increased due to unimproved glucose level. When the situation is aggravated then reaches to insulin resistance, where the blood glucose barely obliged to the insulin. The main symptoms include diabetes by abnormal blood sugar metabolism, an increase of neutral fat by abnormal lipid metabolism, hypercholesterolaemia, hypertension by an increase of sodium, gout by an increase of uric acid, or the like. In general, the condition in which three or more of the five indexes including abdominal obesity, diabetes, hypercholesterolaemia, hypertension, and hypertriglyceridemia exceed baseline values is considered as a metabolic syndrome. However, its exact mechanism is unknown. The ‘metabolic syndrome’ was defined by WHO in 1998. Since reasons for IR mainly include body fat increase by obesity and lack of exercise, rather than congenital reasons, the most effective way to improve IR is weight watching and regular exercise.

Diabetes, representative metabolic syndrome, is a disease which causes a continuous metabolic disorder, such as hyperglycemia, by inadequate insulin function, and thus increases the possibility of vascular complications. The diabetes is roughly divided into insulin-dependent diabetes “type 1 diabetes” and non-insulin-dependent diabetes “type 2 diabetes”.

Currently various diabetes therapeutic agents, recombinant insulin, sulfonylureas, metformin, thiazolidinediones, α-glucosidase inhibitor, etc. are prescribed. Sulfonylureas help beta-cells to secrete insulin even when insulin is not required in the body, and thereby hypoglycemia may be caused (Holman, R. R. et al, Metabolism, 55, S2-5,2006;Pickup, J. C. et al., Blackwell Scientific Publ. London, pp 462-476, 1991). Metformin (a biguanide) inhibits de novo glucose synthesis in liver cells and increases the amount of insulin receptors and is known to cause gastroenteric troubles and toxicity causing death through long term overdose administration (Innerfield, R. J., New Engl. J. Med., 334, pp 1611-1613, 1996). Rosiglitazone (product name: Avandia) and pioglitazone (product name: Actose), which are thiazolidinedione drugs, help insulin action in muscles and adipose tissue, and are very effective in controlling the blood sugar level. However, since they may result in hepatotoxicity as a side effect, a liver function test is regularly required during drug therapy. For example, the sale of Troglitazone, the first thiazolidinedione drug developed, was prohibited due to its hepatotoxicity. Also, thiazolidinedione drugs may cause obesity. An α-glucosidase inhibitor, which has recently become commercially available, primarily delays digestion and absorption of complex carbohydrate in the small intestine, and reduces postprandial hyperglycemia and hyperinsulinemia (characteristics of non-insulin-dependent diabetes). However, the inhibitor causes side effects, such as abdominal swelling, nausea, diarrhea. Also, recently, it has been reported that the inhibitor causes liver damage, and thus a regular liver function test is required during long term administration. Therefore, it is necessary to develop a drug which can minimize the above mentioned side-effects.

In general, obesity accompanies hyperlipemia and increases the probability of developing diabetes. Often high cellular lipid content in the obese causes insulin resistance to insulin sensitive cells. In this context, it is desirable to develop substances which can reduce fat mass as well as blood glucose level, for therapeutic agents for obesity, hyperlipemia and diabetes or the like. The inventors of the present invention found that obovatol and its derivatives activate AMPK, and inhibit synthesis of fat and cholesterol by increasing β-oxidation of fatty acids. Moreover, obovatol and its derivatives have a high blood sugar lowering effect by reducing body fat, and insulin-independent increase of GLUT4 towarding to cell membrane by activating AMPK. Accordingly, the composition of the present invention may be used for foods, drugs, and feeds which are very effective in preventing, inhibiting and treating diabetes.

In accordance with an aspect of the present invention, there is provided a composition for preventing, inhibiting and treating diabetes and metabolic syndrome, which contains obovatol represented by Formula 1 and its derivatives as an active ingredient.

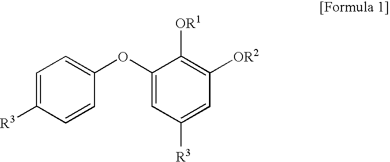

In Formula 1,

R¹ and R² independently or optionally represent hydrogen, C₁-C₇ straight chain or branched-side chain alkyl, acetyl, C₁-C₇ alkoxy or C₁-C₇ alkoxyacetyl; and

R³ represents hydrogen, C₁-C₇ straight chain or branched-side chain alkyl, C₂-C₇ alkenyl or allyl.

Preferably, R¹ and R² independently or optionally represent methyl, ethyl, acetyl, methoxy, ethoxy, methoxyacetyl or ethoxyacetyl; and

R³ represents hydrogen, propyl, propenyl or allyl.

Also, the present invention provides a method of preventing, inhibiting and treating diabetes and metabolic syndrome, the method including the step of administering therapeutically effective amounts of the obovatol and its derivatives to a subject.

Also, the present invention provides a health care food product for preventing, inhibiting and improving diabetes and metabolic syndrome, which contains obovatol and its derivatives as an active ingredient.

Also, the present invention provides an AMP-activated protein kinase(hereinafter, referred to as “AMPK”) enzyme activator containing obovatol and its derivatives as an active ingredient.

The composition containing synthesized obovatol and its derivatives as an active ingredient non-insulin-independently increases a blood sugar lowering effect through the activation of AMPK, and thus may be used as food, medicine, and feed additive which are very effective in preventing, inhibiting and treating diabetes and metabolic syndrome.

Accordingly, the present invention provides a pharmaceutical composition for preventing and treating diabetes and metabolic syndrome, which contains obovatol and its derivatives as a pharmaceutically acceptable active ingredient.

More specifically, the present invention provides a pharmaceutical composition for preventing and treating diabetes and metabolic syndrome through AMPK enzyme activity, which contains obovatol and its derivatives as a pharmaceutically acceptable active ingredient.

Also, the present invention provides an AMPK enzyme activator containing obovatol and its derivatives as a pharmaceutically acceptable active ingredient.

The composition including the compound according to the present invention may further include a carrier, an excipient, or a diluent which is conventionally used in pharmaceutical composition preparation.

The composition including the compound according to the present invention may be prepared in any form, that is, oral forms (such as powder, granule, tablet, capsule, suspension, emulsion, syrup, aerosol.), an external application form, a suppository, or a sterile injection solution according to a conventionally known method. Examples of the carrier, the excipient or the diluent, that may be included in the composition including an extract, may include lactose, dextrose, sucrose, sorbitol, mannitol, xylitol, erythritol, maltitol, starch, acacia senegal gum, alginate, gelatin, calcium phosphate, calcium silicate, cellulose, methyl cellulose, microcrystalline cellulose, polyvinyl pyrrolidone, water, methyl hydroxybenzoate, propyl hydroxylbenzoate, talc, magnesium stearate, mineral oil, or the like. Specifically, in formation, diluent or excipient conventionally known in the art, such as a filler, an extender, a binding agent, a wetting agent, disintegrating agent, a surfactant, may be used in preparation. A solid preparation for oral administration includes tablet, pill, powder, granule, capsule, etc., and such a solid preparation may be prepared by mixing the compound with at least one excipient (such as starch, calcium carbonate, sucrose, lactose, gelatin.). Also, in addition to the excipient, lubricants, such as magnesium stearate, and talc, may be used. A liquid preparation for oral administration includes suspension, liquid for internal use, emulsion, syrup, etc., and in addition to a frequently used main diluent, such as water and liquid paraffin, the preparation may include a variety of excipients (for example, a wetting agent, a sweetening agent, an aromatic agent, a preservative, etc.). A parenteral administration drug includes a sterile aqueous solution, a nonaqueous solvent, suspension, emulsion, a freeze-dried preparation, and a suppository. As the nonaqueous solvent and the suspension, propylene glycol, polyethylene glycol, vegetable oil such as olive oil, injectable ester (such as ethyl oleate), etc. may be used. As a base for the suppository, witepsol, macrogol, tween 61, cacao butter, laurin butter, glycerogelatin, etc. may be used.

Also, the preferable dose of the compound according to the present invention varies according to condition and the weight of a patient, severity of the disease, drug type, administration route and period, etc., and may be appropriately selected by those skilled in the art. In order to obtain a preferable effect, the compound according to the present invention may be administered in amounts of 0.0001 to 100 mg/kg, and preferably 0.001 to 10 mg/kg a day, and may be administered once or several times a day. However, the present invention is not limited to the dose in any ways.

The compound according to the present invention may be administered to mammals such as a rat, a mouse, a domestic animal, a human, through various routes. The administration may be carried out through all possible methods, for example, oral administration, rectal administration, intravenous injection, intramuscular injection, subcutaneous injection, intra-endometrial injection, intracerebroventricular injection. The compound according to the present invention may be pharmaceutically administered as a pharmaceutically acceptable salt thereof, alone or in combination with further another pharmaceutical active compound, but the present invention is not limited thereto.

Also, the present invention provides a health care food for preventing and improving diabetes and metabolic syndrome, which contains the obovatol and its derivatives as an active ingredient as a pharmaceutically acceptable active ingredient, the obovatol and derivatives showing a diabetes preventing/improving effect.

The compound according to the present invention may be variously used for drugs, foods, drinks, etc. for preventing and improving diabetes. The compound according to the present invention may be added to various foods, for example, drinks, gum, tea, a vitamin complex, health promoting foods, etc., and may be used in the form of powders, granules, tablets, capsules, or drinks.

The compound of the present invention hardly causes toxicity and side effects, and thus may be used without anxiety during long-term administration for the purpose of prevention.

The compound of the present invention may be added in foods and drinks for the purpose of preventing and improving diabetes. Herein, in general, a health food composition including the compound may be generally added in an amount of 0.01 to 15 wt % based on the total weight of the food, and a health drink composition including the compound may be added in an amount of 0.02 to 30 g, preferably of 0.3 to 10 g, based on 100 ml.

Besides the compound as an essential ingredient in a predetermined quantity, the health drink composition according to the present invention may include liquid components without particular limitations, and may include various fragrance agents or natural starch, etc. as additives, in the same manner of conventional drinks. Examples of the natural starch include conventional sugar, such as monosaccharide (for example, glucose, fructose, etc.), disaccharide (for example, maltose, sucrose, etc.), polysaccharide (for example, dextrin, cyclodextrin, etc.), and sugar alcohol such as xylitol, sorbitol, erythritol, etc. Also, as the fragrance agent, a natural fragrance agent such as thaumatin, a stevia extract (for example, rebaudioside A, glycyrrhizin, etc.) and a synthetic fragrance agent such as saccharine, aspartame, etc. may be appropriately used. The natural starch is included in an amount of generally 1 to 20 g, and preferably about 5 to 12 g, based on 100 ml of the composition according to the present invention.

In addition, the composition including the compound according to the present invention may contain various nutrients, vitamins, minerals (electrolytes), a flavor agent (such as a synthetic flavor agent, a natural flavor agent, etc.), a coloring agent, an extender (cheese, chocolate, etc.), pectic acid and salt thereof, alginic acid and salt thereof, organic acid, a protective colloid thickener, a PH adjuster, a stabilizer, a preservative, glycerin, alcohol, a carbonating agent used for a carbonated drink, etc. Also, the composition according to the present invention may include flesh that may be used for preparing natural fruit juice, fruit juice drinks, and vegetable drinks. Each of such ingredients may be used independently or in any combination thereof. Although the percentages of such additives are not important, the additives are generally selected in the range of about 0 to 20 parts by weight, based on 100 parts by weight of the composition according to the present invention.

Also, the present invention provides an animal feed additive which is useful for preventing and improving diabetes, and a feed including the same, the animal feed additive containing obovatol and its single derivatives as a pharmaceutically acceptable active ingredient, the obovatol and its derivatives showing a diabetes/ metabolic syndrome preventing and improving effect.

The animal feed additive compound may be presented in the form of highly concentrated liquid (20 to 90%), powders or granules.

The animal feed additive compound according to the present invention may further include one or more materials selected from the group including organic acids(such as citric acid, fumaric acid, adipic acid, lactic acid, malic acid) phosphates (such as sodium phosphate, potassium phosphate, sodium acid pyrophosphate, polyphosphate), and natural antioxidants (such as polyphenol, catechin, alpha-tocopherol, rosemary extract, vitamin C, green-tea extract, licorice extract, chitosan, tannic acid, phytic acid).

The animal feed additive containing the compound according to the present invention, and the feed containing the same may include, as auxiliary substances, various adjuvants, such as amino acid, mineral, vitamin, antibiotic substance, antibacterial substance, antioxidant/antifungal enzymes, living microbial agents. Herein, the adjuvants may be used together with cereals, for example, crushed or cracked wheat, oat, barley, corn and rice; vegetable protein feed including rapeseeds, beans, and sunflower, as principle components; animal protein feed, for example blood meal, meat meal, bone meal and fish meal; and sugar and dairy products, which is obtained by mixing dried components and dried additives (such as, milk powders and whey powders), and then adding liquid components, and lipid (to be liquefied through heating), for example, animal fats and vegetable fats optionally liquefied by heating. In addition, the adjuvants may further include nutrition supplements, a digestion-absorption enhancer, a growth promoting agent, a disease preventing agent, or the like.

The animal feed additive compound may be administered alone or in combination with another feed additive in an edible carrier. Also, the animal feed additive compound may be used as a top dressing material, or directly mixed with animal feed. Otherwise, apart from the feed, the compound may be easily given by oral administration, injection or transdermal application, or may be used in combination with other components. In general, as known in the art, a daily dosage amount may be administered once or several times a day.

When the animal feed additive compound is administered apart from animal feed, the compound may be prepared into instant or sustained release preparations by combination with a non-toxic pharmaceutically acceptable edible carrier as known in the art. Such an edible carrier may be solid or liquid, for example, cornstarch, lactose, sucrose, bean flakes, peanut oil, olive oil, sesame oil and propylene glycol. In the case of the solid carrier, the dosage form of the compound may be tablet, capsule, powder, troche, saccharated tablet or micro-dispersion type top-dressing. In the case of liquid carrier, the dosage form of the compound may be soft gelatin capsule, syrup, liquid suspension, emulsion or solution. Also, such various administration forms may include an adjuvant, for example, a preservative, a stabilizer, a wetting agent, an emulsifier, a solution promoting agent, or the like.

Also, the animal feed including the compound of the present invention as an additive may include protein-containing organic cereal powder conventionally used for satisfying dietary needs of animals. Such protein-containing organic cereal powder usually mainly includes corns, bean powder or corn/bean mixed powder.

The animal feed additive may be added to the animal feed through immersion, spray or mixing. The present invention may be applied to foods of various animals including mammals, poultry, fishes. More specifically, the feed may be used for commercially important mammals, for example, pigs, cows, sheep, goats, laboratory rodents (rats, mice, hamsters and gerbils), furry animals (for example, minks and foxes), zoo animals (for example, monkeys and apes), and livestock (for example, cats and dogs). In general, commercially important poultry includes chickens, turkeys, dogs, geese, pheasants and quails, and may also include raising fish such as trout.

In blending the animal feed including the compound according to the present invention, the compound is mixed with animal feed in an amount of about 1 g to 100 g based on 1 kg of feed (dried weight). After the mixing is completed, a caking material which is pelletized or granular according to crushed extent of the components is obtained. The material may be provided as mash, or may be formed into a desired separate type for further processing or packaging. Herein, in order to prevent feed materials from falling off during storage, it is preferable to add water to the animal feed, and then to subject the feed to conventional processes, such as pelletization, expansion, or extrusion. Excess water may be dried and removed.

The composition according to the present invention, which contains obovatol and its derivatives as an active ingredient, activates an AMPK enzyme, and thus can improve insulin resistance and non-insulin-dependently increase a high blood sugar lowering effect through AMPK activation. Thus, it can be used for foods, drugs, and feed additives which are very effective in preventing, inhibiting and treating diabetes and metabolic syndrome.

### BRIEF DESCRIPTION OF THE DRAWINGS

The above and other objects, features and advantages of the present invention will be more apparent from the following detailed description taken in conjunction with the accompanying drawings, in which:

FIG. 1 shows NMR spectrum of tetrahydro obovatol (ob-Rd);

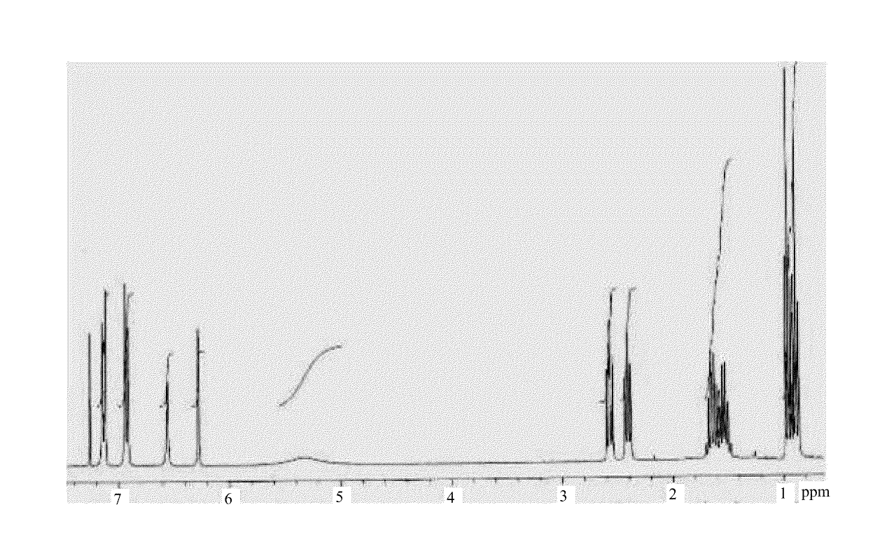

FIG. 2 shows activation increase of AMPK in mouse-derived L6 muscle cells according to treatment concentration of test samples, which was carried out by immunostaining based on phosphorylation increase of a threonine residue in position 172 of AMPK's alpha subunit;

FIG. 3 shows glucose uptake increase within cells when L6 muscle cells are treated with test samples for 2 hours; and

FIG. 4 shows the change in the blood sugar level when test samples were orally administered to diabetes model db/db mice.

### DETAILED DESCRIPTION OF THE EXEMPLARY EMBODIMENTS

Hereinafter, the present invention will be described with reference to Examples and Experimental Examples. However, the following examples are illustrative only, and the scope of the present invention is not limited thereto.

### Referential Example 1

### Reagents and Devices

An analysis device—¹H-NMR (400 MHz) and ¹³C-NMR (400 MHz) spectrometer (JNM-AL 400, JEOL Ltd., Japan), a melting pointer (Yamako, MD-S3, Japan), a mass spectrometer (MS, PE SCIX API 2000 MS/MS, Canada) were used. As reagents, products from Aldrich Chemical Co. were used, and as other solvents, reagents (at least first grade) were used without purification. In order to purify synthesized materials, Silica gel (Merck, 230-400 mesh) was used.

### Example 1

### Preparation of Obovatol

To 1 kg of dried Japanese magnolia fruit, 4 L of methanol or ethanol was added, and the fruit was left at room temperature for 5 days. An organic solvent layer was separated, vacuum-dried, adsorbed onto silica gel and eluted with an active fraction by hexane and ethyl acetate (in a ratio of 9:1). Then, from the 1 kg of dried Japanese magnolia fruit, 40 g of liquid obovatol was obtained.

### Example 2

### Preparation of Tetrahydro Obovatol (ob-Rd)

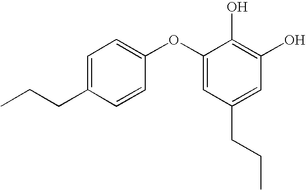

1 g of obovatol obtained from Example 1 was dissolved in acetone (200), and then at room temperature, 2 mg of palladium-charcoal (Pd-Charcoal) was added thereto. Then, the resulting mixture was hydrogenated.

Then, an organic solvent layer containing an active ingredient was separated and vacuum-concentrated. The concentrated extract (1.1 g) was dissolved in methylene chloride(100), and was added to silica gel (Merck, Art No. 9385) to adsorb the active ingredient onto silica gel. Next, while the ratio of ethyl acetate to hexane was increased from 10:90 to 20:80, an active fraction was eluted via silica gel column chromatography to provide 0.9 g of colorless ob-Rd (yield: 90%).

### Example 3

### Preparation of 3-(4-propylphenoxy)-2-methoxy-5-propylphenol (ob-Rd-2M)

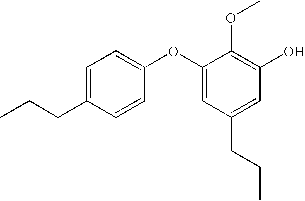

1 g of ob-Rd obtained from Example 2 was dissolved in acetone(200), and potassium carbonate(K₂CO₃) (1.1 g) and methyliodide(0.3 g) were added thereto, followed by stirring at room temperature for 5 hours. After the reaction has completed, an organic solvent layer containing an active ingredient was separated and vacuum-concentrated. The concentrated extract (1.2 g) was dissolved in methylene chloride(30), and was added to silica gel (Merck, Art No. 9385) to adsorb the active ingredient onto silica gel. Next, while the ratio of ethyl acetate to hexane was increased from 10:90 to 20:80, an active fraction was eluted via silica gel column chromatography to provide colorless 3⁻(4⁻propylphenoxy)-2-methoxy-5-propylphenol(ob-Rd-2M, 1 g) (yield: 90%).

### Example 4

### Preparation of 1-(4-propylphenoxy)-2,3-diacetyloxy-5-propylbenzene (ob-Rd-Ac)

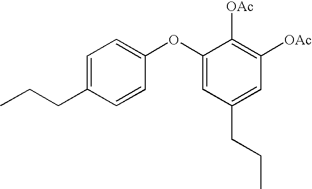

Colorless 1-(4-propylphenozy)-2,3-diacetyloxy-5-propylbenzene (1.1 g) was prepared in the same manner as described in Example 3, except that ob-Rd (1 g)obtained from Example 2 was dissolved in acetone(200), and potassium carbonate(K₂CO₃) (1.2g) and acetyl chloride (500 mg) were added thereto, followed by stirring at room temperature for 5 hours, (yield: 90%).

### Example 5

### Preparation of 3-(4-((E)-prop-1-enyl)phenoxy)-5-((E)-prop-1-enyl)benzene-1,2-diol (ob-RE)

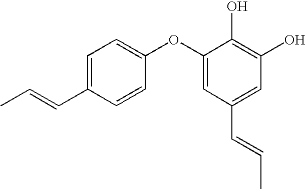

1 g of obovatol was dissolved in methanol, and palladium chloride (PdCl₂) (10 mg) was added thereto, followed by stirring at room temperature for 5 hours. After the reaction is completed, an organic solvent layer containing an active ingredient was separated and vacuum-concentrated. The concentrated extract (1.1 g) was dissolved in methylene chloride(30), and was added to silica gel (Merck, Art No. 9385) to adsorb the active ingredient onto silica gel. Next, while the ratio of ethyl acetate to hexane was changed to 20:80, an active fraction was eluted via silica gel column chromatography. Then, by drying the active fraction, colorless 3-(4-((E)-prop-1-enyl)phenoxy)-5-((E)-prop-1-enyl)benzene-1,2-diol (0.91g) was obtained (yield: 90%).

### Example 6

### Preparation of 1-(4-((E)-prop-1-enyl)phenoxy)-2,3-di-2-methoxyacetyloxy-5-((E)-prop-1-enyl)benzene (ob-RE-MAc)

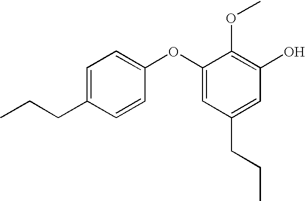

Colorless 1-(4-((E)-prop-1-enyl)phenoxy)-2,3-di-2-methoxyacetyloxy-5-((E)-prop-1-enyl)benzene(1.15 g) was prepared in the same manner as described in Example 2, except that propenyl obovatol (1 g) obtained from Example 5 was dissolved in acetone(200), and potassium carbonate(K₂CO₃) (1.2 g) and methoxyacetyl chloride (600mg) were added thereto, followed by stirring at room temperature for 5 hours, (yield: 90%).

### Example 7

### Preparation of 1-(4-((E)-prop-1-enyl)phenoxy)-2,3-diacetyloxy-5-((E)-prop-1-enyl)benzene (ob-RE-Ac)

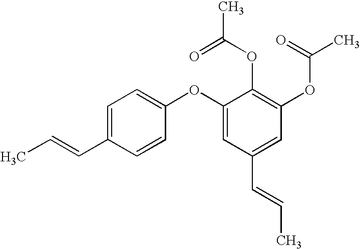

Colorless 1-(4-((E)-prop-1-enyl)phenoxy)-2,3-diacetyloxy-5-((E)-prop-1-enyl)benzene (1.0 g) was obtained in the same manner as described in Example 2, except that propenyl obovatol (1 g) obtained from Example 5 was dissolved in acetone (200), and potassium carbonate(K₂CO₃) (1.3 g) and acetyl chloride (500 mg) were added thereto, followed by stirring at room temperature for 5 hours, (yield: 90%).

[Analysis]

Each of the compounds prepared by Examples 1 to 7 were subjected to UV absorbance analysis, IR(infrared) absorbance analysis and high resolution mass spectrometry analysis so as to determine the molecular weight and formula of the purified compound. Specifically, UV absorbance analysis was carried out by UV-265 spectrophotometer (Shimadzu), IR absorbance analysis was carried out by FTS-80 spectrophotometer (Bio-Rad Digilab Division), and the molecular weight and formula were determined through High resolution MS analysis by using VG70-SEQ mass spectrometry. Also, a nuclear magnetic resonance device (Varian 300, 500 NMR) was used to obtain ¹H, ¹³C-NMR spectrum, and the spectrum was synthetically analyzed to determine the structure. Tables 1 and 2 show the results of the analysis.

TABLE 1
Appearance, molecular formula, molecular weight and solubility

| | | Molecular | Molecular | Solubility | |
|---|---|---|---|---|---|
| Index | Color | formula | weight | Soluble | Insoluble |
| Example | Colorless | C₁₈H₁₈O₃ | 282 | Alcohol, | Water |
| 1 | | | | DMSO | |
| Example | Colorless | C₁₈H₂₂O₃ | 286 | Alcohol, | Water |
| 2 | | | | DMSO | |
| Example | Colorless | C₁₉H₂₄O₃ | 300 | Alcohol, | Hexane, |
| 3 | | | | DMSO | Water |
| Example | Colorless | C₂₂H₂₆O₃ | 370 | Alcohol, | Water |
| 4 | | | | DMSO | |
| Example | Colorless | C₁₈H₁₈O₃ | 282 | Alcohol, | Water |
| 5 | | | | DMSO | |
| Example | Colorless | C₂₄H₂₆O₇ | 426 | Alcohol, | Water |
| 6 | | | | DMSO | |
| Example | Colorless | C₂₂H₂₂O₅ | 366 | Alcohol, | Water |
| 7 | | | | DMSO | |

TABLE 2
NMR data

| Index | NMR data |
|---|---|
| Example | ¹H NMR (300 MHz, CDCl₃): 7.15 (2H, d, J = 9 Hz), 6.94 |
| 1 | (2H, d, J = 9 Hz), 6.59 (1H, d, J = 1.5 Hz), 6.30 |
| | (1H, d, J = 1.5 Hz), 5.97 (2H, m), 5.06 (4H, m), 3.38 |
| | (2H, d, J = 6.6 Hz), 3.20 (2H, d, J = 6.6 Hz) ppm |
| Example | ¹H-NMR (CDCl₃): 7.13 (2H, d, J = 8.7 Hz), 6.95 (2H, d, |
| 2 | J = 8.7 Hz), 6.57 (1H, d, J = 1.5 Hz), 6.3 (1H, d, J = |
| | 2.1 Hz), 2.58 (2H, t, J = 8.1 Hz), 2.42 (2H, t, J = |
| | 8.1 Hz), 1.59 (4H, m), 0.92 (6H, m) ppm. |
| Example | ¹H-NMR (CDCl₃): 7.15 (2H, d, J = 9 Hz), 6.97 (2H, d, J = |
| 3 | 9 Hz), 6.66 (1H, d, J = 1.5 Hz), 6.37 (1H, d, J = |
| | 1.5 Hz), 3.92 (3H, s), 2.61 (2H, t, J = 8.1 Hz), 2.48 |
| | (2H, t, J = 8.1 Hz), 1.65 (4H, m), 0.97 (6H, m) ppm. |
| Example | ¹H-NMR (CDCl₃): 7.12 (2H, d, J = 8.7 Hz), 6.92 (2H, d, |
| 4 | J = 8.7 Hz), 6.74 (1H, d, J = 2.1 Hz), 6.63 (1H, d, J = |
| | 2.1 Hz), 2.56 (2H, t, J = 8.1 Hz), 2.48 (2H, t, J = |
| | 8.1 Hz), 2.29 (3H, s), 2.19 (3H, s), 1.59 (4H, m), |
| | 0.92 (6H, m) ppm. |
| Example | ¹H-NMR (CDCl₃): 7.28 (2H, d, J = 9 Hz), 6.95 (2H, d, J = |
| 5 | 9 Hz), 6.73 (2H, d, J = 1.5 Hz), 6.42 (2H, d, J = |
| | 1.5 Hz), 6.37 (1H, d, J = 15.5 Hz), 6.15 (2H, m), |
| | 5.99 (1H, m), 5.42 (2H, s), 1.88 (3H, d, J = 7 Hz), |
| | 1.80 (3H, d, J = 7 Hz). |
| Example | ¹H-NMR (CDCl₃): 7.27 (2H, d, J = 8.1 Hz), 6.95 (2H, d, |
| 6 | J = 8.1 Hz), 6.77 (1H, d, J = 1.5 Hz), 6.17 (5H, m), |
| | 4.27 (2H, s), 4.14 (2H, s), 3.525 (3H, s), 3.41 (3H, |
| | s), 1.88 (3H, d, J = 6.6), 1.83 (3H, d, J = 6.6). |
| Example | ¹H-NMR (CDCl₃): 7.27 (2H, d, J = 8.1 Hz), 6.97 (2H, d, |
| 7 | J = 8.1 Hz), 6.88 (1H, d, J = 1.5 Hz), 6.75 (1H, d, J = |
| | 1.5 Hz), 6.37 (1H, d, J = 14.7 Hz), 6.14 (3H, m), |
| | 2.29 (3H, s), 2.18 (3H, s), 1.87 (3H, d, J = 6.6 Hz), |
| | 1.81 (3H, d, J = 6.6 Hz). |

### Experimental Example 1

### Effect on AMPK Activation

In order to test AMPK enzyme activation by compounds synthesized from Examples 1 to 7, the effects of the compounds were measured by using a method described in the reference (Schlattner U, et al, J Biol Chem. 279, pp 43940-51, 2004).

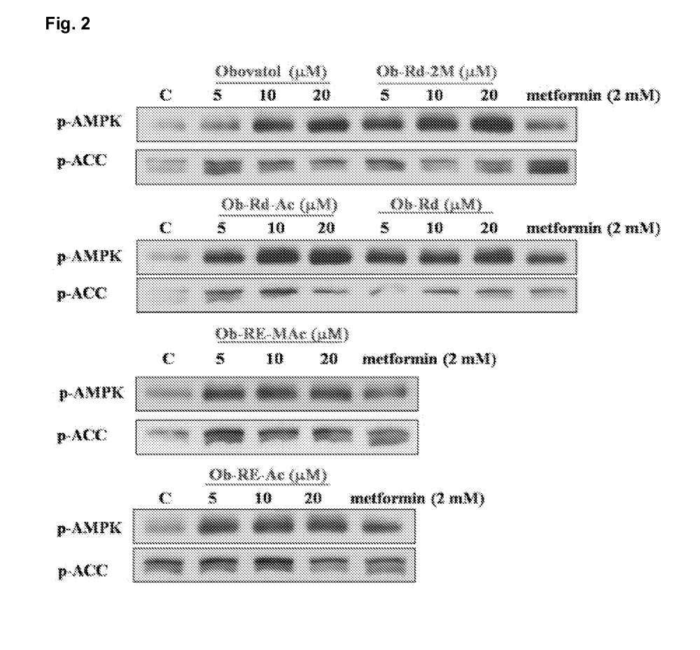

AMPK antibodies (Cell Signaling Technology, Beverly, Mass.) in L6 myotube cell (American Type Culture Collection, Manassas Va.) in activated state, which were treated with Obovatol and its derivatives with different concentrations (5, 10, and 20 μM) as shown in FIG. 2, were prepared. In order to examine the activation increase of AMP-activated protein kinase (hereinafter, referred to as “AMPK”) enzyme according to treatment concentrations, phosphorylation increase of a threonine residue at position 172 of AMPK alpha subunit was analyzed through western blot assay as shown in FIG. 2.

As a result, as shown in FIG. 2, all of the obovatol and its single derivatives showed increasing tendencies as concentration increased. From the results, it can be seen that the obovatol and its derivatives are advantageous in AMPK activation.

### Experimental Example 2

### Test on Glucose Uptake in L6 Myotube Cells

In order to measure glucose uptake in L6 myotube cells through treatment of obovatol and its single derivatives synthesized from Examples 1 to 6, the effects of the compounds were measured (Somwar R et al, Clin Ther. 20, 125-40, 1998).

Completely differentiated L6 myotube cells were cultured in a serum-free culture medium (GIBCO, Auckand NZ) for 6 hours, and treated with obovatol and its single derivatives, respectively, by 10 μM, for 2 hours, and then reacted in HEPES-saline buffer containing 10 μM unlabeled 2-deoxyglucose, and 10 μM 2-deoxy-[³H]-glucose (1 mCi/ml) for 10 minutes. Then, the reacted cells were washed with ice-cold PBS three times, and were subjected to lysis by using 1N NaOH. Scintillation counter (Packard Co. 1600PR) was used to measure cpm.

Herein, non-specific glucose uptake within the cells was measured by using HEPES-saline buffer containing 10 μM cytochalasin B(Sigma Chemical Co. St Louis Mo.) and then was subtracted from the total value.

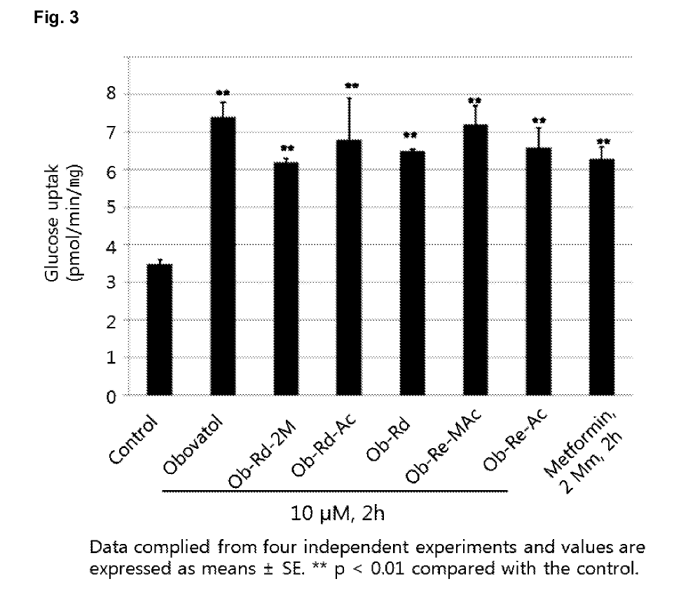

As a result, as shown in FIG. 3, it can be seen that glucose uptake was increased by the treatment of obovatol and its single derivatives with the same concentration.

Experimental Example 3

### Measurement of Blood Glucose Level in db/db Mice

In order to measure the blood glucose level in db/db mice through treatment of the compounds obtained from Examples, the effects of the compounds were measured by using a method described in the reference (Halseth A E et al, Biochem Biophys Res Commun. 294, 798-850, 2002).

Sixteen diabetic db/db mice (5 weeks old, The Jackson Laboratory, Bar Harbor, ME, Inc Germany) were divided into two groups, and then to each of the groups, obovatol with concentrations of 5 and 10 mg/kg (olive oil) was orally administered for 4 weeks. Meanwhile, to a control group, the same volume of saline solution orally administered for 4 weeks. Then, the change in blood glucose level of the mice was recorded.

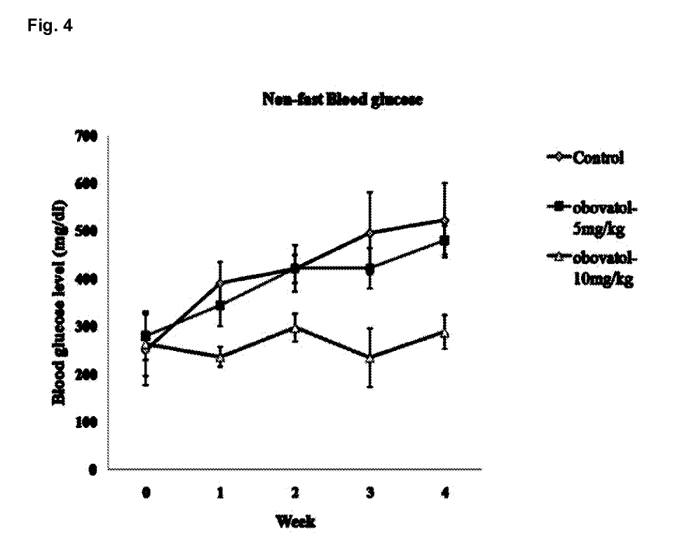

As a result, as shown in FIG. 4, the administation of 5 mg/kg obovatol did not show a significant change in blood glucose level, compared to the control group, while the administation of 10 mg/kg obovatol showed significantly reduced blood glucose levels, compared to the control group (the reduced extent: after 1 week, 39.6% (235.75±20.9, 390.25±44.1), after 2 weeks, 29.2% (297.25±29, 419.75±28.8), after 3 weeks, 52.7% (234±61.5, 495±86.2), and after 4 weeks, 55.2% (288±27.6, 522±78)).

### Experimental Example 4

### Test on Acute Toxicity

In accordance with guidelines of Korea Food and Drug Administration (KFDA), a test on acute toxicity of the compound according to the present invention was carried out by using male ICR mice (4 weeks old, Jung Ang Lab. Animal Inc).

To each group including 10 mice, the compound of the present invention was orally administered in an amount of 0.625˜2 g/kg once, and observation was carried out for 2 weeks.

After the administration, mortality rate, clinical symptoms, and weight changes of animals were observed, and blood-biochemical analysis was carried out. Through autopsy, whether abnormality of organs and thoracic organs exists or not was observed with the naked eye.

As a result, from among the animals to which experimental materials were injected, not one showed specific clinical symptoms. In addition, there were no dead animals, and also toxicity was not shown in the observation of weight change, a blood test, blood-biochemical analysis, and autopsy.

It was determined that the compound of the present invention does not show toxicity when orally administered to a mice in an amount of up to 2 g/kg. Also, it was determined that the compound is effective when orally administered to a human body in an amount of 1-1000 mg/kg, and when transdermally administered in an amount of 0.2-100 mg/cm2.

Hereinafter, formulation examples of the composition are illustrative, and the scope of the present invention is not limited thereto.

### Preparation Example 1

### Preparation of Pharmaceutical Formulations

1-1. Preparation of Powders

| | |
|---|---|
| compound by Example 1 | 2 g |
| lactose | 1 g |

The above noted ingredients were mixed and filled into sealed packaging to provide powders.

1-2. Preparation of a Tablet

| | |
|---|---|
| compound by Example 1 | 100 mg |
| cornstarch | 100 mg |
| lactose | 100 mg |
| stearic acid | 2 mg |
| magnesium | |

The above noted ingredients were mixed and tabletted according to a conventional tablet preparation method to provide a tablet.

1-3. Preparation of a Capsule

| | |
|---|---|
| compound by Example 1 | 100 mg |
| cornstarch | 100 mg |
| lactose | 100 mg |
| stearic acid | 2 mg |
| magnesium | |

The above noted ingredients were mixed and filled into a gelatin capsule according to a conventional capsule preparation method to provide a capsule.

1-4. Preparation of a Pill

| | |
|---|---|
| compound by Example 1 | 1 g |
| lactose | 1.5 g |
| Glycerin | 1 g |
| xylitol | 0.5 g |

The above noted ingredients were mixed and prepared into a pill according to a conventional method in such a manner that one pill has a weight of 4 g.

1-5. Preparation of a Granule

| | |
|---|---|
| compound by Example 1 | 150 mg |
| Soybean extract | 50 mg |
| Glucose | 200 mg |
| starch | 600 mg |

The above noted ingredients were mixed and 100 mg of 30% ethanol was added thereto, followed by drying at 60° C. After formation of granules, the granules were filled into packaging.

### Preparation Example 2

### Preparation of a Food

Foods containing the synthesized compound of the present invention were prepared by the following processes.

2-1. Preparation of a Cooking Spice

A cooking spice⁻for health care was prepared by using 20 to 95 parts by weight of the compound by Example 2.

2-2. Preparation of Tomato Ketchup and Sauce

Health care tomato ketchup or sauce was prepared by adding 0.2 to 1.0 parts by weight of the compound by Example 3 to tomato ketchup or sauce.

2-3. Preparation of a Flour Food

Health care flour foods (for example, breads, cakes, cookies, crackers, and noodles) were prepared by adding 0.5 to 5.0 parts by weight of the compound by Example 4 to flour, and using the mixture.

2-4. Preparation of Soup and Gravies

Health care meat products, soup (for noodles), and gravies were prepared by adding 0.1 to 5.0 parts by weight of the compound by Example 5 to soup or gravies.

2-5. Preparation of Ground Beef

Health care ground beef was prepared by adding 10 parts by weight of the compound by Example 6 to ground beef.

2-6. Preparation of Dairy Products

Various dairy products (such as butter and ice cream) were prepared by adding 5 to 10 parts by weight of compound by Example 2 to milk, and using the milk.

2-7. Preparation of Sunsik (Powdered Food Steamed and Parched with Various Grains)

Brown rice, barley, glutinous rice, adlay were pregelatinized, dried, and then roasted according to a conventionally known method. Then, the grains were prepared into powders with a grain size of 60 mesh by using a crusher.

Black soybean, black sesame, perilla seeds were steamed, dried and then roasted according to a conventionally known method. Then, the seeds were prepared into powders with a grain size of 60 mesh by using a crusher.

The compound by Example 3 was vacuum-concentrated in a vacuum concentrator, and dried by spray and a hot wind dryer. Then the resulting dried product was prepared into dried powders with a grain size of 60 mesh by using a crusher.

The grains, the seeds and the dried powders of the compound by Example 3 were blended with each other at the following ratio.

grains (30 parts by weight of brown rice, 15 parts by weight of adlay, 20 parts by weight of barley),

seeds (7 parts by weight of perilla seeds, 8 parts by weight of black soybean, 7 parts by weight of black sesame),

dried powders of the compound by Example 1 (3 parts by weight),

ganoderma lucidum (0.5 parts by weight),

rehmannia glutinosa (0.5 parts by weight)

2-8. Preparation of a Health Care Food

| one of compounds by Examples 1 to 6 | 1000 | mg |
|---|---|---|
| vitamin mixture | proper quantity | |
| vitamin A acetate | 70 | μg |
| vitamin E | 1.0 | mg |
| vitamin B₁ | 0.13 | mg |
| vitamin B₂ | 0.15 | mg |
| vitamin B₆ | 0.5 | mg |
| vitamin B₁₂ | 0.2 | μg |
| vitamin C | 10 | mg |
| biotin | 10 | μg |
| nicotinic acid amid | 1.7 | mg |
| Folic acid | 50 | μg |
| calcium pantothenate | 0.5 | mg |
| inorganic mixture | proper quantity | |
| ferrous sulfate | 1.75 | mg |
| zinc oxide | 0.82 | mg |
| magnesium carbonate | 25.3 | mg |
| potassium phosphate monobasic | 15 | mg |
| calcium phosphate dibasic | 55 | mg |
| potassium citrate | 90 | mg |
| calcium carbonate | 100 | mg |
| magnesium chloride | 24.8 | mg |

In the above composition including vitamins and minerals, the ingredients are mixed in a ratio appropriate for a health care food, but the mixing ratio may be changed. A health care food composition may be prepared according to a conventional method of preparing a health care food, the method including the steps of mixing the above ingredients, preparing granules, and using the granules in the same manner as the conventional method.

### Preparation Example 3

### Preparation of a Drink

3-1. Preparation of a Health Care Drink

| | | |
|---|---|---|
| one of compounds by Examples 1 to 6 | 1000 | mg |
| citric acid | 1000 | mg |
| oligosaccharide | 100 | g |
| plum concentrate | 2 | g |
| taurine | 1 | g |

| | |
|---|---|
| Purified water | To total volume |
| | of 900 ml |

According to a conventional production method of a health care drink, the above ingredients are mixed and are agitation-heated for about 1 hour at 85° C.; the resultant solution is filtered and is fed into a 21 sterilized container, and the solution is subjected to a sealing and sterilizing process and is kept refrigerated. Then, the final solution is used for preparing the health care drink composition according to the present invention.

In the above composition, the ingredients are mixed in a ratio appropriate for a health care drink, but the mixing ratio may be changed according to regional/national preferences, such as classes, nations, purposes of consumers.

3-2. Preparation of Vegetable Juice

5 g of one of the compounds by Examples 1 to 6 was added to 1,000 ml of tomato or carrot juice to provide a health care vegetable juice.

3-3. Preparation of Fruit Juice

1 g of one of the compounds by Examples 1 to 6 was added to 1,000 ml of apple or grape juice to provide a health care fruit juice.

Although an exemplary embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A composition for inhibiting, improving or treating diabetes, comprising an effective amount of the compound represented by Formula 1 or a pharmaceutically acceptable salt thereof as an active ingredient: 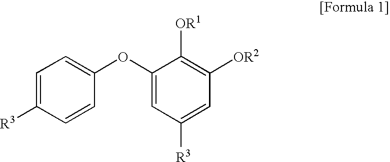
wherein R¹ and R² independently represent hydrogen, C₁-C₇ straight chain or branched-side chain alkyl, acetyl, C₁-C₇ alkoxy or C₁-C₇ alkoxyacetyl; and
R³ represents hydrogen, C₁-C₇ straight chain or branched-side chain alkyl, C₂-C₇ alkenyl or allyl;
wherein, when both R¹ and R² are hydrogen, at least one R³ is not allyl.

2. The composition as claimed in claim 1, which is in the form of drink, powder, granule, tablet or capsule.

3. The composition of claim 1, wherein the effective amount of the compound is 0.01 to 15 wt % based on the total weight of the composition.

4. The composition of claim 1, wherein the effective amount of the compound is 0.02 to 30 g based on 100 ml of the composition.

5. The composition of claim 1, wherein the administration of the composition to a patient increases activity of an AMP-activated protein kinase(AMPK) enzyme.

6. A health care food comprising the composition of claim 1.

7. A method for inhibiting, improving or treating diabetes, comprising administering to a subject in need thereof an effective amount of the compound represented by Formula 1 or a pharmaceutically acceptable salt thereof as an active ingredient: 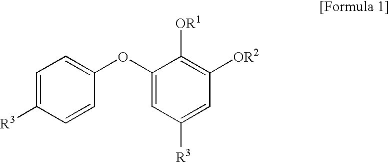
wherein R¹ and R² independently or optionally represent hydrogen, C₁-C₇ straight chain or branched-side chain alkyl, acetyl, C₁-C₇ alkoxy or C₁-C₇ alkoxyacetyl; and
R³ represents hydrogen, C₁-C₇ straight chain or branched-side chain alkyl, C₂-C₇ alkenyl or allyl.

8. The method of claim 7, wherein the effective amount of the compound is about 0.0001 to 100 mg/kg per day.

9. The method of claim 8, wherein the effective amount of the compound is about 0.001 to 10 mg/kg per day.

10. The method of claim 7, wherein the compound is comprised in a health care food.

11. The method of claim 10, wherein the health care food is in the form of drink, powder, granule, tablet or capsule.

12. A method for increasing activity of an AMP-activated protein kinase(AMPK) enzyme comprising administering to a subject in need thereof an effective amount of the compound represented by Formula 1 or a pharmaceutically acceptable salt thereof as an active ingredient: 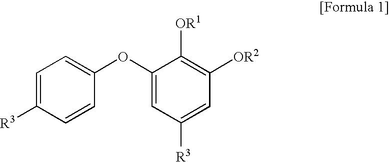
wherein R¹ and R² independently or optionally represent hydrogen, C₁-C₇ straight chain or branched-side chain alkyl, acetyl, C₁-C₇ alkoxy or C₁-C₇ alkoxyacetyl; and
R³ represents hydrogen, C₁-C₇ straight chain or branched-side chain alkyl, C₂-C₇ alkenyl or allyl.
